(19) United States
(12) Patent Application Publication
(10) Pub. No.: US 2008/0109922 A1
(43) Pub. Date: May 8, 2008

(54) Coated seeds and materials for seed coating

(75) Inventor: Long-Shyong Wang, Taoyuan (TW)

Correspondence Address: BIRCH STEWART KOLASCH & BIRCH, PO BOX 747, FALLS CHURCH, VA, 22040-0747, US

(73) Assignee: Watack Chemical Company

(21) Appl. No.: 11/592,292

(22) Filed: Nov. 3, 2006

(51) Int. Cl. A01H 5/10; C08G 69/00; C08G 59/00; C08G 63/00

(52) U.S. Cl. 800/298; 525/88; 525/420; 528/361; 528/87

(57) ABSTRACT

The present invention relates to a warm melt polymer and a plant seed coated with the warm melt polymer. The warm melt polymers are fabricated from a reaction of a carboxylic or epoxy-containing water insoluble polymer and a hydroxyl containing water soluble polymer. The warm melt polymer is highly resistant to embitterment and dry crumbling and adheres well to the seeds. The seeds coated with the warm melt polymers of the present invention have excellent storage properties and would not prematurely begin germination during the course of storage.

### BACKGROUND OF THE INVENTION

1. Field of Invention

The present invention relates to the plant seeds coated with warm melt properties materials. More specifically, the present invention relates to the materials with warm melt properties used for seed coating.

2. Background of the Art

The coating of plant seeds has been long known and practiced. It represents an additional expense in materials and processing, but offers a variety of individual or combined advantages that outweigh the expense. Plant seeds coating operations involve the use of polymer coatings to delay germination of the seeds or coating with particulate materials for one or more beneficial purposes. For example, coating seeds renders them more resistant to water and wind erosion and makes them easier to plant. By including pH modifiers or nutrients in the coating, the resulting seeds can be made more favorable for the germination and growth of the resulting plants.

However, the known coating process have numerous shortcomings. It has been general practice to coat seeds with water-based water-soluble polymers, i.e. methyl cellulose, gum Arabic and starch, with the intent that the coating would disintegrate or dissolve rapidly in contact with water such that germination was delayed minimally. There have been, however, limitations in employing water-soluble polymers. First, applications, which are water-based, have moisture in the coating process, which causes premature germination during the coating process. Second, such coatings require relatively high temperatures to remove water and that may kill the seeds. Third, coatings do not adhere to nor properly coat seeds. This is evident in those seed, which exhibit rapid water uptake. During the coating operation, the seeds absorb the water found in traditional coating and considerable swelling is frequently observed. After drying, the seeds retract to their original size and owing to the brittleness of traditional coatings, the coating no longer adhere properly to the seeds negating the protective and growth promoting properties originally intended.

Precise sowing either mechanically or by hand has recently come into general use in order to save labor and stabilized growth in vegetable culture. Further, transplanting culture using a seedling tray, pot, etc. has become popular, too. With respect to the sowing into a seedling tray, it is said that accurate and precise sowing by machines or instruments is a necessary condition for growing of good seedlings. However, precise sowing is very difficult to achieve mechanically in the case of odd-shaped seeds of lettuce, carrot, and Welsh onion and in the case of small-grained seed of cabbage and Chinese cabbage. Hand sowing of these seeds is inefficient; requiring much labor to overcome this disadvantage. Hand or mechanical sowing has another disadvantage of having to remove extra or over crowded seeds to obtain proper spacing between two seeds. Also it is necessary to remove uncultivated weeds for desired plants to grow. It has become a common practice to coat the seeds with a proper material to produce spherical grains larger than the seeds and deposit these coated seeds on a moving paper roll with proper spacing between the seeds.

The method as defined in U.S. Pat. No. 4,879,839 coats seeds with low melting point temperature polyesters. Specifically the process consists of treating the seed by means of a solution of polyester in an organic solvent or by means of using polyester in the liquid or molten state. The polyesters have a low melting point below 60° C. The polyesters are chosen from homopolymers and copolymers of epsilon caprolactone. However, they do not possess warm melt adhesive properties where by the adhesive to be reheated again for further application.

The method defined in U.S. Pat. No. 5,044,116 coats seeds with a layer of low melting point polyester and a peroxy compound to improve the characteristics of germination and to provide various additives facilitating the establishment and growth of plants. The process involves treating the seeds with a peroxy compound in the presence of a solution of the polyester in an organic solvent or with the polyester in the liquid or melted state. This method also does not possess any warm melt adhesive properties after the seeds were coated.

Similar materials in U.S. Pat. No. 5,552,519 stated a maleic anhydride adducted rosin ester reacted with polyethylene glycol (PEG) of molecular weight of 8000 formed a surfactant which was used as a surfactant to disperse rosin ester into water. Also in U.S. Pat. No. 6,274,657B1 shown dimmer rosin reacted with PEG with molecular weight of 8000 form a product. The product can be used as a surfactant to disperse rosin ester or hydrocarbon resin into water.

The present invention attempts to develop the seed coating materials which can be heated and partially re-melted again at relatively low temperatures to allow the seeds to be stick to and deposited on a moving bio-degradable paper roll at various pre-designated spacing. The paper serves the additional function of preventing weed growth that which would compete with seeds for soil nutrients.

### SUMMARY OF THE INVENTION

One aspect of the present invention is aimed at providing the coated seeds with excellent storage properties due to the high adhesive property of the coating material. The seed coating does not prematurely start germination during the storage. When seeds are sown in their cultivation medium, the coating materials also do not produce a toxic effect on either the seeds, the resulting plant or soil.

Another aspect of the present invention is aimed at providing the coating materials, which can be coat the plant seeds at relatively low melting temperatures and are not tacky at ambient temperatures. Any unused part of the coating in the pipeline can be recycled by simply re-melting and collection operations. Also their desirable low-temperature re-melting properties would allow the embedding of the coated seeds onto a paper roll for pre-determined spacing. Proper spacing between the seeds ensures efficient use of the seeds by preventing over crowding.

To achieve the above goals, the present invention provides a plant seed coated with warm melt polymer, comprising: a plant seed having surface; and a warm melt polymer coating on said surface, wherein the warm melt polymer containing at least one ester group is fabricated from a reaction of a carboxylic or epoxy-containing water insoluble polymer and a hydroxyl-containing water soluble polymer; and said warm melt polymer has a warm melt adhesive property between 50° C.-70° C.

The present invention also provides a warm melt polymer containing at least one ester group fabricated from a reaction of a carboxylic or epoxy-containing water insoluble polymer and a hydroxyl-containing water soluble polymer, wherein said carboxylic-containing water insoluble polymer is fabricated from a reaction of an unsaturated carboxylic acid or an anhydride compound with a non-carboxylic-containing polymer. Said non-carboxylic-containing polymer is selected from a group consisting of C5 hydrocarbon resin, C9 hydrocarbon resin, C5 and C9 hydrocarbon copolymer, Terpene resin, terpene styrene copolymer, terpene phenolic resin, alpha pinene maleic anhydride alternating copolymer, and mixture thereof.

The coating polymers of the present invention are highly resistant to embrittlement and dry crumbling and adhere well to the seeds. The coating materials will dissolve under the effect of moisture or water when the seeds are sown in their cultivation medium. They do not produce a phytoxic effect and thus avoid premature germination of the seeds.

The seeds coated with the warm melt polymers of the present invention have excellent storage properties and would not prematurely germinate during storage. Furthermore, the coating of the coated seeds re-melt at relative low temperature would allow them to embed the coated seeds onto a paper roll at a pre-determined spacing.

### DETAIL DESCRIPTION OF THE PRESENT INVENTION

In one embodiment, the warm melt polymer of the present invention is fabricated from a reaction of a carboxylic-containing water insoluble polymer and a hydroxyl water soluble polymer. The warm melt polymer has a warm melt adhesive property between 50° C.-70° C.

The hydroxyl water soluble polymer comprises polyoxyethylene glycol (PEG), polyoxyethylene polyoxypropylene glycol block copolymer.

The carboxylic-containing water insoluble polymer comprises rosin, dimmer rosin, polymeric rosin or mixture thereof.

The hydroxyl water soluble polymer may be employed alone or in combination. The hydroxyl water soluble polymer is preferably employed in such an amount that the ratio of the hydroxyl equivalents of the hydroxyl water soluble polymer to the carboxyl equivalents of the rosin is in the range from 0.5 to 2.

The carboxylic-containing water insoluble polymer also can be fabricated from a reaction of an unsaturated carboxylic acid or an anhydride compound with a non-carboxylic-containing polymer.

The unsaturated carboxylic acid or anhydride compound comprises fumaric acid and its half esters, maleic acid, its anhydride and its half esters, acrylic acid, methyl acrylic acid, aryl acid, itaconic acid and its anhydride, oligomers and copolymers of acrylics and vinyl with ethylenically unsaturated acid, or styrene/acrylic acids copolymer, and fumaric acid or maleic anhydride are preferable.

The non-carboxylic-containing polymer comprises rosin ester, C5 hydrocarbon resin, C9 hydrocarbon resin, C5 and C9 hydrocarbon copolymer, terpene resin, terpene styrene copolymer, terpene phenolic resin, alpha pinene and maleic anhydride alternating copolymer, or mixture thereof.

In another embodiment, the warm melt polymer of the present invention fabricated from a reaction of an epoxy-containing water insoluble polymer and a hydroxyl-containing water soluble compound, has a warm melt adhesive property between 50° C.-70° C.

Preferably, the epoxy-containing polymer is epoxy bisphenol A, and the hydroxyl-containing polymer comprises PEG or polyoxyethylene polyoxypropylene glycol block copolymer.

The warm melt polymers disclosed in above embodiments have a molecular weight of 2,000 to 25,000.

As the warm melt polymers disclosed above are used for seed coating, the present invention also discloses a plant seed coated with warm melt polymer, comprises: a plant seed having surface, and a warm melt polymer coating on said surface. The warm melt polymer is fabricated from a reaction of a carboxylic or epoxy-containing water insoluble polymer and a hydroxyl-containing water soluble polymer, and the warm melt polymer has a warm melt adhesive property between 50° C.-70° C.

In preferred embodiment, the warm melt polymer coated on the plant seed has a molecular weight of 2,000 to 25,000.

The hydroxyl-containing water soluble polymer comprises PEG or polyoxyethylene polyoxypropylene glycol block copolymer.

In one embodiment, the carboxylic-containing water insoluble polymer comprises rosin, dimmer rosin, polymeric rosin or mixture thereof.

In another embodiment, the carboxylic-containing water insoluble polymer is fabricated from a reaction of an unsaturated carboxylic acid or an anhydride compound with a non-carboxylic-containing polymer. The unsaturated carboxylic acid or anhydride compound comprises fumaric acid and its half esters, maleic acid, its anhydride and its half esters, acrylic acid, methyl acrylic acid, aryl acid, itaconic acid and its anhydride, oligomers and copolymers of acrylics and vinyl with ethylenically unsaturated acid, or styrene/acrylic acids copolymer, and fumaric acid or maleic anhydride are preferably.

The non-carboxylic-containing polymer comprises rosin ester, C5 hydrocarbon resin, C9 hydrocarbon resin, C5 and C9 hydrocarbon copolymer, terpene resin, terpene styrene copolymer, terpene phenolic resin, alpha pinene maleic anhydride alternating copolymer, or mixture thereof.

The epoxy-containing water insoluble polymer is, but not limited to epoxy bisphenol A.

The weight of the water insoluble polymer is 1% to 35% of water soluble compound in the warm melt polymer.

The reaction of the carboxylic-containing polymer and the hydroxyl-containing water soluble polymer including the reaction ratios can be referred, but not limited, as follows: (1) Rosin ester having an acid value of less than 20; (2) Grafting the rosin ester from (1) with an unsaturated carboxylic acid or anhydride, the weight ratio of carboxylic acid or anhydride/rosin ester is from 1% to 30%; (3) Grafting of the hydrocarbon resins with an unsaturated carboxylic acid or anhydride, the weight ratio carboxylic acid or anhydride to hydrocarbon resin is from 1 to 30%; (4) The carboxylic acid group reacted with PEG of a molecular weight between 2,000 and 20,000. The molar ratio of free carboxylic acid group/glycol is about 1 to 4 and the reaction product will have an acid value of less than 2 after reacted; and (5) Rosin, dimmer rosin, polymeric rosin or their mixture with an unsaturated carboxylic acid reacted with a polyethylene glycol of a molecular weight from 2,000 to 20,000. The molar ratio of free carboxylic acid group/glycol is about 1 to 4 and the reaction product will have an acid value of less than 2 after reacted.

According above, the plant seeds coating materials can be prepared from rosin. The term “rosin” is used herein and in the claims is referred to rosin, rosin dimmer or mixture of rosin unless otherwise noted. Rosin is a natural products consisting of unsaturated acids. The rosin acids are mainly monobasic carboxylic acid containing 20 carbon atoms in the molecules. Natural rosin may be classified due to its origin, namely as gum rosin, tall oil rosin and wood rosin. The plant seed-coating materials according to this invention can be prepared from any of these rosins or from mixtures thereof.

According to this invention, the rosin ester obtained from esterified rosin is grafted with an unsaturated carboxylic acid or anhydride. Preferably the carboxylic acid is an alpha-beta ethylenically unsaturated acid which is grafted onto the rosin by reaction with unsaturated part of the rosin. A Diels-Alder adduction is formed from a conjugated double bond of the rosin acid and alpha-beta unsaturated carboxylic acid or anhydride.

The unsaturated carboxylic acid is reacted in the molar ratio of 0.1 to 1. preferably 0.3 to 0.6 with each rosin associated with rosin ester. i.e., pentaerythritol rosin ester has a maximum of 4 rosins associated with it and a glycerol ester has maximum of 3. The final acid value should at least be greater than 20. The reaction temperature is from about 170° C. to about 240° C.).

The esterifying catalysts employed if desired are acidic catalysts such as sulfuric acid, hypophosphorous acid and p-toluene sulfonic acid.

The grafted rosin ester is further esterified with a PEG with a molecular weight of 2,000-20,000. The molecular weight of the PEG is preferably 4,000 to 10,000, more preferably 4000-8000. PEG is suitable reacted in the molar ratio 0.5 to 2.0 with carboxylic group, so that the final acid value is less than 10, preferably 5, and most preferably 2. The reaction is suitably undertaken at a temperature of between 180° C. to 280° C., preferably at 265° C. to 275° C., and from 1 hour to 20 hours in the presence of a catalyst. Suitable catalysts include acidic catalysts such as sulfuric acid, hypophosporous acid, and p-toluene sulfonic acid.

The total dose of coating can vary within very wide limits depend on the type of the seeds, their shape, and their size. The total dose of coating is generally at least 10% of the weight of the seeds. Most frequently, this dose does not exceed 10 times the weight of the seeds. When it is unnecessary to give the coated seeds a predetermined shape or size, the coating dose is most frequently at least 10% and preferably at least 50% of the weight of the seeds. Generally it does not exceed 300% and preferably does not exceed 200% of the weight.

The dose of polyesters in the coating according to this invention can be variable. They can be from 0.1% to 100% of the total weight of the coating. The concentration of polyesters can be from 10% to 100% of the weight of the coating excluding the fillers. It is often more than 50% by weight and preferably 80% to 100% by weight.

The other additives can vary within very wide limits. It is in general 0% to 100% of the total weight of the coating and preferably between 0.1% to 80% of the total weight of the coating.

Particularly advantageous coating can thus contain 80% to 100% by weight of polyesters according to the invention and from 0% to 80% of various additives. The coating is present in the quantities of 0.1% to 100% of the weight of the uncoated seeds.

The invention may be applied to various types of seeds especially vegetable seeds including root vegetable seeds, stem vegetable seeds, Leaf vegetable seeds, flower vegetable seeds, fruit vegetable seeds and flower seeds. The paper sheet are spread on the soil, after sprinkling water morning and afternoon, all seeds are sprout-within three days. With 99.5% sprout rate were realized.

The present invention relates to seed coating with an anhydrous coating which has been applied to the seeds in an anhydrous melt and according to which the coating contains polyester having a low melting temperature. The melting temperature does not exceed over 70° C., and preferably does not excess 68° C. The polyester chosen is preferably from those with melting temperatures below 65° C.

According to this invention, the coating operation may be carried out according to various methods. The polyester is employed in a molten state. When needed, fillers or other additives are also added. The polyester in the molten state is applied to the seeds and then the fillers or other additives are added. The addition of polyester in the molten state may take place during the addition of fillers or other additives. The addition of fillers or other additives may be interrupted once or several times and new application of polyester in the molten state may be made in the interval. This being done until the seeds has been completely coated.

The present invention also relates to a process for reheating the coated material at relatively low temperatures from 50° C. to 70° C., preferably from 55° C. to 68° C. and most preferably from 55° C. to 65° C. The reheated coated seeds will stick onto the rolling paper moving at a relatively high speed, often at 30 meter per minute.

The paper roll with the coated seeds should be kept in a dry place with low humidity or in a vacuum bag for transportation. It can be keep intact for many years in dry environmental condition. The seeds roll can be laid on soil when the season is right. The seeds would normally start the germination within a few days after being sprinkled with water.

The seed coating operation according to this invention, the seed coating material is melted and directly coated onto the seeds at relatively low temperatures without using any water or solvent. This coated material is tack free after coating and cooling immediately. It is highly resistant to brittleness and to dry crumbling.

After the coating operation is completed, the coated material on the seeds can be reheated again at low temperature and the seeds may adhere onto the paper seeds carrier. This unique warm melt adhesive property has not been found on any of the previous patents.

The present invention relates to the coating materials for the seeds. These coating materials can be prepared with different solubility in water for the purpose of controlling the water absorption rate when planting.

The coating may contain various additives. Thus it may contain fillers of an organic type. Mixtures of fillers may also be incorporated in it. The fillers are fine powder and preferably pass through a 325 mesh sieve (U.S. Standard). Frequently, fillers are of organic type of natural products such as cornstarch powder. Fillers, such as ground talc, silica, calcium carbonate and their mixtures can also be used.

The coating may also contain one or more other additives such as plant protection materials, such as insecticides, fungicides, disinfectants, herbicides and growth regulators, agents for the protection against the harmful effects of the selective herbicides (such as activated charcoal, nutrients), agents which is capable of improving germination and product quality, bacteria which is capable of beneficially affecting the germination, the formation or growth of plants and the like.

The doses of polyester in the coating according to this invention depend on the type of seeds, their shape, and their size. The total dose of coating is generally at least 1% of the weight of the seeds. Most frequently, this does not exceed 10 times the weight of the seeds. The dose limit is normally not critical.

The following examples are used to further demonstrate the advantages of the present invention and to expand rather than limit its scope.

### EXAMPLE

### Example 1

### Preparation of PEG Ester from Rosin

10 grams of rosin with acid number of 180 was charged into a 2 liters three necks flask. 385 grams of PEG (with a molecular weight of 8000) was added to the flask. The reactants in the flask were reacted at 275° C. under a nitrogen atmosphere for a period of 3 hours. 0.2% based on the total reactants weight of 50% hypophosphorus acid catalyst was added into reaction flask slowly. The contents were held at 275° C. for 5 more hours. The resulting condensation product had an acid number less then 2. The PEG ester of the polymeric rosin had a softening point of 58 C. as determined by differential scanning calorimetry.

### Example 2

### Preparation of PEG Ester from Polymeric Rosin

15 grams of polymeric rosin containing about 60-70% of dimmer rosin with acid number of 140 was charged into a three necks flask. 199 grams of PEG (with a molecular weight of 8000) was added to the flask. The reactants in the flask were reacted at 275° C. under a nitrogen atmosphere for a period of 3 hours. 0.2% based on the total reactants weight of 50% hypophosphorus acid catalyst was added into reaction flask slowly. The contents were held at 275° C. for 5 more hours. The resulting condensation product had an acid number less then 2 and this PEG ester of the polymeric rosin had a softening point of 59.5° C. as determined by differential scanning calorimetry.

### Example 3

### Preparation of PEG Ester from Rosin Ester

A 1 liter three neck flask equipped with a temperature controller, overhead stirrer, heating mantle, and condenser is charged with 100 grams of rosin ester (softening point 90° C.) and this rosin ester is heated to 180° C. Then 10 grams of maleic anhydride powder is slowly added to the flask. After the addition of maleic anhydride, the temperature is raised and maintained at 240° C. for four hours. Any unreacted maleic anhydride is removed by purging with nitrogen for half an hour, while maintaining the temperature at 240° C. The resulting maleated rosin ester had a softening point of 142° C. and acid value of 63. A 20 grams of this product is further reacted with 270 grams of PEG with a molecule weight of 8000 at 275° C. in the presence of 1.5 gram of 50% hypophosphorous acid for 12 hours or until the acid value was less then 4 and the product had a softening point of 59° C.

### Example 4

### Preparation of PEG Ester from Maleic Adducted Rosin

A 2 liter three neck flask equipped with a temperature controller, overhead stirrer, heating mantle and condenser is charged with 100 grams of rosin (softening point 76° C.) and this rosin was heated to 180° C. Then 6 grams of maleic anhydride powder is slowly added to the flask. After the addition of maleic anhydride, the temperature is raised and maintained at 240° C. for four hours. Any unreacted maleic anhydride is removed by purging with nitrogen for half an hour while maintaining the temperature at 240° C. The resulting maleated rosin has a softening point of 86° C. and acid value of 204. With 20 grams of this product is further reacted with 300 grams of PEG with a molecule weight of 4000 at 275° C. in the presence of 1.6 gram of 50% hypophorous acid for 12 hours or until the acid value becomes less then 2 and the product has a softening point of 55° C.

### Example 5

### Preparation of PEG Ester from Maleic Anhydride Adducted C5 Hydrocarbon Resin

A 2 liter three neck flask equipped with a temperature controller, overhead stirrer, heating mantle and condenser is charged with 100 grams of a C5 hydrocarbon resin (softening point 70° C.) and this hydrocarbon resin was heated to 160° C. then 4.5 grams of maleic anhydride powder is slowly added to the flask. After the addition of maleic anhydride, the temperature is raised and maintained at 240° C. for four hours. Any unreacted maleic anhydride is removed by purging with nitrogen for half an hour while maintaining the temperature at 240° C. The resulting maleated C5 hydrocarbon resin has an acid value of 56. With 20 grams of this product is further reacted with 160 grams of PEG with a molecule weight of 8000 at 275° C. in the presence of 0.9 gram of 50% hypophorous acid for 12 hours or until the acid value becomes less then 2 and the product has a softening point of 56.5° C.

### Example 6

### Preparation of PEG Ester from Maleic Anhydride Adducted Terpene

A 2 liter three neck flask equipped with a temperature controller, overhead stirrer, heating mantle and condenser is charged with 100 grams terpene resin (softening point 67.5° C.) and this terpene resin was heated to 180° C. 9 grams of maleic anhydride powder is slowly added to the flask. After the addition of maleic anhydride, the temperature is raised and maintained at 240° C. for four hours. Any unreacted maleic anhydride is removed by purging with nitrogen for half an hour while maintaining the temperature at 240° C. The resulting maleated terpene resin has an acid value of 94 and softening point of 86° C. With 20 grams of this product is further reacted with 400 grams of PEG with a molecule weight of 8000 at 275° C. in the presence of 2.1 gram of 50% hypophorous acid for 12 hours or until the acid value becomes less then 2 and the product has a softening point of 59° C.

### Example 7

### Preparation of PEG Ester from Maleic Anhydride Adducted Terpene Styrene Copolymer

A 2 liter three neck flask equipped with a temperature controller, overhead stirrer, heating mantle and condenser is charged with 100 grams terpene styrene copolymer (softening point 73° C.) and this terpene styrene copolymer was heated to 180° C. 3 grams of maleic anhydride powder is slowly added to the flask. After the addition of maleic anhydride, the temperature is raised and maintained at 240° C. for four hours. Any unreacted maleic anhydride is removed by purging with nitrogen for half an hour while maintaining the temperature at 240° C. The resulting maleated terpene resin has an acid value of 21 and softening point of 80° C. With 20 grams of this product is further reacted with 600 grams of PEG with a molecule weight of 8000 at 275° C. in the presence of 3.1 gram of 50% hypophorous acid for 12 hours or until the acid value becomes less then 2 and the product has a softening point of 59° C.

### Example 8

### Preparation of PEG Ester from Maleic Adducted Terpene Phenolic Resin

A 2 liter three neck flask equipped with a temperature controller, overhead stirrer, heating mantle and condenser is charged with 100 grams terpene phenolic resin (softening point 70° C.) and this terpene resin was heated to 180° C. 7 grams of maleic anhydride powder is slowly added to the flask. After the addition of maleic anhydride, the temperature is raised and maintained at 240° C. for four hours. Any unreacted maleic anhydride is removed by purging with nitrogen for half an hour while maintaining the temperature at 240° C. The resulting maleated terpene phenolic resin has an acid value of 72 and softening point 91° C. 20 grams of this product is further reacted with 200 grams of PEG with a molecule weight of 8000 at 275° C. in the presence of 1.1 gram of 50% hypophorous acid for 12 hours or until the acid value becomes less then 2 and the product has a softening point of 59° C.

### Example 9

### Preparation of PEG Ester from Alpha Pinene Maleic Anhydride Alternating Copolymer

A 2 liter three neck flask equipped with a temperature controller, overhead stirrer, heating mantle and condenser is charged with 20 grams alpha pinene maleic anhydride alternating copolymer (softening point 128° C. and molecular weight 984) was reacted with 230 grams of PEG with a molecule weight of 8000 at 275° C. in the presence of 1.2 gram of 50% hypophorous acid for 12 hours or until the acid value becomes less then 2 and the product has softening point of 58° C.

### Example 10

### Preparation of PEG Ester from Epoxy

A 2 liter three neck flask equipped with a temperature controller, overhead stirrer, heating mantle and condenser is charged with 28 grams Epoxy Bisphenol A and 800 grams of PEG with a molecule weight of 8000 at 275° C. in the presence of 4 gram of 50% hypophorous acid for 12 hours or until the acid value becomes less then 2 and the product has softening point of 58° C.

### Example 11

### Vegetable Seeds Coated with the PEG Ester from Example 1

Vegetable seeds are coated by molten polymeric PEG esters from example 1 in a coating apparatus. The PEG esters is heated to molten state and sprayed onto the seeds in a rotating coating apparatus. As the spraying molten rosin ester reached the surface of the vegetable seeds the temperature was less then 45° C. After it adhered onto the surface of the seeds, the PEG esters is solidified on the seeds surface and become tack free. Other ingredients can be multi-coated on the seeds by powder coating or molten coating layer by layer. The coating weight is about 100% of original seeds weight.

The coated seeds are mechanically pre-arranged on a roller and dropped onto a roll of paper. As soon as these coated seeds reach the surface of the paper, the coated seeds are heated instantaneously by a heating device under the paper. These coated seeds then become adhered to the paper immediately. The coated seeds adhered on the paper are then subjected to −40° C. cooling before being rolled onto a jumbo roll for future application.

What is claims is:

1. A plant seed coated with warm melt polymer, comprising:
a plant seed having surface; and
a warm melt polymer coating on said surface,
wherein said warm melt polymer containing at least one ester group is fabricated from a reaction of a carboxylic or epoxy-containing water insoluble polymer and a hydroxyl water soluble polymer; and said warm melt polymer has a warm melt adhesive property between 50° C.-70° C.

2. The plant seed according to claim 1, wherein said warm melt polymer has a molecular weight of 2,000 to 25,000.

3. The plant seed according to claim 1, wherein said hydroxyl water soluble polymer comprises polyethylene glycol or polyoxyethylene polyoxypropylene glycol block copolymer.

4. The plant seed according to claim 1, wherein said carboxylic-containing water insoluble polymer comprises rosin, dimmer rosin, polymeric rosin or mixture thereof.

5. The plant seed according to claim 1, wherein said carboxylic-containing water insoluble polymer is fabricated from a reaction of an unsaturated carboxylic acid or an anhydride compound with a non-carboxylic-containing polymer.

6. The plant seed according to claim 5, wherein said unsaturated carboxylic acid or anhydride compound comprises fumaric acid and its half esters, maleic acid, its anhydride and its half esters, acrylic acid, methyl acrylic acid, aryl acid, itaconic acid and its anhydride, oligomers and copolymers of acrylics and vinyl with ethylenically unsaturated acid, or styrene/acrylic acids copolymer.

7. The plant seed according to claim 6, wherein said unsaturated carboxylic acid or anhydride compound is fumaric acid or maleic anhydride.

8. The plant seed according to claim 5, wherein said non-carboxylic-containing polymer comprises rosin ester, C5 hydrocarbon resin, C9 hydrocarbon resin, C5/C9 hydrocarbon copolymer, terpene resin, terpene styrene copolymer, terpene phenolic resin, alpha pinene maleic anhydride alternating copolymer, or mixture thereof.

9. The plant seed according to claim 1, wherein said epoxy-containing water insoluble polymer is epoxy bisphenol A.

10. The plant seed according to claim 1, wherein the weight of said water insoluble polymer is 1% to 35% of water soluble polymer in said warm melt polymer.

11. A warm melt polymer containing at least one ester group fabricated from a reaction of a carboxylic or epoxy-containing water insoluble polymer and a hydroxyl water soluble polymer, wherein said carboxylic-containing water insoluble polymer is fabricated from a reaction of an unsaturated carboxylic acid or an anhydride compound and a non-carboxylic-containing polymer, said non-carboxylic-containing polymer is selected from a group consisting of hydrocarbon resin, terpene resin, terpene styrene copolymer, terpene phenolic resin, alpha pinene maleic anhydride alternating copolymer, and mixture thereof.

12. The warm melt polymer according to claim 11, wherein said hydroxyl water soluble polymer comprises polyethylene glycol or polyoxyethylene polyoxypropylene glycol block copolymer.

13. The warm melt polymer according to claim 11, wherein said epoxy-containing polymer is epoxy bisphenol A.

14. The warm melt polymer according to claim 11, wherein said unsaturated carboxylic acid or anhydride compound comprises fumaric acid and its half esters, maleic acid, its anhydride and its half esters, acrylic acid, methyl acrylic acid, aryl acid, itaconic acid and its anhydride, oligomers and copolymers of acrylics and vinyl with ethylenically unsaturated acid, or styrene/acrylic acids copolymer.

15. The warm melt polymer according to claim 13, wherein said unsaturated carboxylic acid or anhydride compound is fumaric acid or maleic anhydride.

16. The warm melt polymer according to claim 11, wherein said warm melt polymer has a molecular weight of 2,000 to 25,000.

17. The warm melt polymer according to claim 11, wherein said hydrocarbon resin is C5 hydrocarbon resin, C9 hydrocarbon resin, or C5/C9 hydrocarbon copolymer.
